(19) United States
(12) Patent Application Publication
(10) Pub. No.: US 2011/0190252 A1
(43) Pub. Date: Aug. 4, 2011

(54) COMPOSITIONS INCLUDING CLAVULANIC ACID AND RELATED METHODS OF USE

(76) Inventors: Alan Watson, Lexington, MA (US); Manuel A. Navia, Boston, MA (US)

(21) Appl. No.: 13/056,565

(22) PCT Filed: May 8, 2009
(86) PCT No.: PCT/US09/43289

§ 371 Date: Apr. 25, 2011

Related U.S. Application Data
(60) Provisional application No. 61/084,910, filed on Jul. 30, 2008.

(51) Int. Cl. A61K 31/43; A61K 31/424; A61K 31/431; A61P 11/00

(52) U.S. Cl. 514/192; 514/210.06

(57) ABSTRACT

Methods of treating a disorder in a subject comprising administering to a subject an elastase inhibitor such as clavulanic acid, sulbactam, or tazobactam are described.

### BACKGROUND

Clavulanic acid (CVA), sulbactam, and tazobactam are mechanism-based inhibitors of the serine-dependent bacterial β-lactamases, enzymes that promote antibiotic resistance through the hydrolysis of the reactive β-lactam group in penicillins and cephalosporins.

### SUMMARY

The inventors have discovered novel methods of using clavulanic acid, sulbactam, and tazobactam and related compositions. For example, the inventors have discovered that an elastase inhibitor such as clavulanic acid, sulbactam, or tazobactam can be used to treat a disorder relating to a serine protease (e.g., Cathepsin G, elastase, and Protease 3). In some embodiments, the disorder is characterized by unwanted serine protease activity. In some preferred embodiments, clavulanic acid, sulbactam, or tazobactam (e.g., clavulanic acid) can be used to treat disorders related to increased elastase activity (e.g., increased activity in the lung). Increased elastase activity can be the result of recruitment of neutrophils to a site of inflammation, for example, inflammation as the result of a disease exacerbation, such as an infection, an environmental insult, or an allergic or other inflammatory response.

Inhibition of elastase can prevent the destruction of natural defense mechanisms in the lung—a process orchestrated by the pathogens, which can pervert the neutrophil response to infection to inappropriately liberate neutrophil elastase. An inhibitor of elastase can be used to prevent the destruction of naturally occurring defense mechanisms. For example, an inhibitor of elastase can reduce the severity of an infection and/or prevent the establishment or reduce the severity of a chronic infection in, for example, a subject suffering from cystic fibrosis or COPD. Other exemplary disorders that can be treated with an elastase inhibitor such as clavulanic acid, sulbactam, or tazobactam include emphysema, ARDS, SIRS, asthma, and ALI. In some preferred embodiments, clavulanic acid, sulbactam, or tazobactam is used to treat cystic fibrosis or an underlying condition in a subject suffering from cystic fibrosis.

In some embodiments, the elastase inhibitor such as clavulanic acid, sulbactam, or tazobactam can be administered with an additional therapeutic agent, such as an anti-infective or an anti-inflammatory agent. In some embodiments, the elastase inhibitor such as clavulanic acid, sulbactam, or tazobactam or a composition comprising an elastase inhibitor such as clavulanic acid, sulbactam, or tazobactam will be administered in a composition suitable for inhaled administration.

In one aspect, the invention features a method of treating a disorder in a subject, the method including administering to a subject an inhibitor of an elastase such as clavulanic acid, sulbactam, or tazobactam.

In some embodiments, the disorder is a disorder related to excess elastase activity. In some embodiments, the disorder is an inflammatory disorder, for example, an inflammatory condition of the lung such as cystic fibrosis (for example, persistence of infection in a cystic fibrosis patient, for example pseudomonas or other gram negative infection), emphysema (for example, genetic AAT deficiency, COPD (e,g., lung degeneration in chronic bronchitis, lung degeneration in asthma, and other COPD), acute lung injury (ALI), acute respiratory distress syndrome (ARDS), systemic inflammatory response syndrome (SIRS). In some embodiments, condition is ALI and the ALI is caused by systemic inflammatory response syndrome (SIRS). In some embodiments, the disorder is a bronchial disorder such as bronchiectasis. In some embodiments, the disorder is a pulmonary disorder, for example, a chronic pulmonary disorder such as inflammation or cystic fibrosis. In some embodiments, the disorder is an acute pulmonary disorder, for example, including inflammation. In some embodiments, the acute pulmonary disorder is acute respiratory distress syndrome.

In some embodiments, the method also includes administering an additional therapeutic agent, for example, an anti-infective or an anti-inflammatory agent. Exemplary antibiotics include gram negative, gram positive, and broad spectrum antibiotics. Antibiotics can be characterized into the following classifications. Aminoglycosides; beta-lactam antibiotics such as Carbacephems, Carbapenems, Cephalosporins, and Penicillins; Macrolides; antibiotic polypeptides; antibiotic lipopeptides (e.g., daptomycin); antibiotic glycopeptides; Monobactams; Quinolones; Sulfonamides; Tetracyclines; and others. Preferred antibiotics are those effective in treating gram negative infections. Exemplary preferred gram negative antibiotics include tobramycin and aztreonam. In some preferred embodiments, the antibiotic is formulated to provide desirable lung retention or residence time.

Exemplary antibiotics within the classes recited above are provided as follows. Exemplary Aminoglycosides include Streptomycin, Neomycin, Framycetin, Parpmycin, Ribostamycin, Kanamycin, Amikacin, Dibekacin, Tobramycin, Hygromycin B, Spectinomycin, Gentamicin, Netilmicin, Sisomicin, Isepamicin, Verdamicin, Amikin, Garamycin, Kantrex, Netromycin, Nebcin, and Humatin. Exemplary carbacephems include Loracarbef (Lorabid). Exemplary carbapenems include ertapenem, invanz, doripenem, finibax, imipenem/cilastatin, primaxin, meropenem, and merrem. Exemplary cephalosporins include cefadroxil, Duricef, cefazolin, ancef, cefalotin, cefalothin, keflin, cefalexin, Keflex, Cefaclor, Ceclor, Cefamandole, Mandole, Cefoxitin, Mefoxin, Cefprozill, Cefzil, Cefuroxime, Ceftin, Zinnat, Cefixime, Suprax, Cefdinir, Omnicef, Cefditoren, Spectracef, Cefoperazone, Cefobid, Cefotaxime, Claforan, Cefpodoxime, Fortaz, Ceftibuten, Cedax, Ceftizoxime, Ceftriaxone, Rocephin, Cefepime, Maxipime, and Ceftrobriprole. Exemplary glycopeptides include dalbavancin, oritavancin, Teicoplanin, Vancomycin, and Vancocin. Exemplary macrolides include Azithromycin, Sithromax, Smamed, Zitrocin, Clarithromycin, Biaxin, Dirithromycin, Erythromycin, Erythocin, Erythroped, Roxithromycin, Troleandomycin, Telithromycin, Ketek, and Spectinomycin. Exemplary monobactams include Aztreonam. Exemplary penicillins include Amoxicillin, Novamox, Aoxil, Apicillin, Alocillin, Crbenicillin, Coxacillin, Diloxacillin, Flucloxacillin Floxapen, Mezlocillin, Meticillin, Nafcillin, Oacillin, Penicillin, and Ticarcillin. Exemplary polypeptides include Bacitracin, Colistin, and Polymyxin B. Exemplary quiniolones include Ciproflaxin, Cipro, Cproxin, Ciprobay, Enoxacin, Gatifloxacin, Tequin, Levofloxacin, Levaquin, Lomefloxacin, Moxifloxacin, Avelox, Norfloxacin, Noroxin, Ofloxacin, Ocuflox, Trovafloxacin, and Trovan. Exemplary sulfonamides include Mefenide, Prontosil (archaic), Sulfacetamide, Sulfamethizole, Sulfanilamide (archaic), Sulfasalazine, Sulfisoxazole, Trimethoprim, Trimethoprim-Sulfamethoxazole (co-trmoxazole), and Bactrim. Exemplary tetracyclines include Demeclocyline, Doxycycline, Vibramycin, Minocycline, Minocin, Oxytetracycline, Terracin, Tetracycline, and Sumycin. Other exemplary antibiotics include rshenamine, Salvarsan, Chloamphenicol, Chloromycetin, Clindamycin, Cleocin, Linomycin, Ethambutol, Fosfomycin, Fusidic Acid, Fucidin, Furazolidone, Isoniazid, Linezolid, Zyvox, Metronidazole, Flagyl, Mupirocin, Bactroban, Nitrofurantion, Macrodantin, Macrobid, Platensimycin, Pyrazinamide, Quinupristin/Dalfopristin, Syncerid, Rifampin (rifampicin), and Timidazole.

In some embodiments, the inhibitor of an elastase such as clavulanic acid, sulbactam, or tazobactam is administered in a composition that is substantially free of amoxicillin and ticarcillin.

In one aspect, the invention features a method of treating a condition or disorder triggered by a neutrophil response. The method includes administering to a subject an inhibitor of an elastase such as clavulanic acid, sulbactam, or tazobactam.

In some embodiments, the disorder is a disorder related to excess elastase activity. In some embodiments, the disorder is an inflammatory disorder, for example, an inflammatory condition of the lung such as cystic fibrosis (for example, persistence of infection in a cystic fibrosis patient, for example pseudomonas or other gram negative infection), emphysema (for example, genetic AAT deficiency, COPD (e,g., lung degeneration in chronic bronchitis, lung degeneration in asthma, and other COPD), acute lung injury (ALI), acute respiratory distress syndrome (ARDS), systemic inflammatory response syndrome (SIRS). In some embodiments, condition is ALI and the ALI is caused by systemic inflammatory response syndrome (SIRS). In some embodiments, the disorder is a bronchial disorder such as bronchiectasis. In some embodiments, the disorder is a pulmonary disorder, for example, a chronic pulmonary disorder such as inflammation or cystic fibrosis. In some embodiments, the disorder is an acute pulmonary disorder, for example, including inflammation. In some embodiments, the acute pulmonary disorder is acute respiratory distress syndrome.

In some embodiments, the neutrophil response causes the release of elastase in the pancreas. In some embodiments, the disorder is pancreatitis.

In some embodiments, the inhibitor of an elastase such as clavulanic acid, sulbactam, or tazobactam is administered via an inhaled formulation. In some embodiments, the inhaled formulation includes a composition having particles, and wherein the mean diameter of the particles is from about 0.1 μm to about 50 μm (e.g., from about 0.2 μm to about 10 μm). In some embodiments the particles are a solid (e.g., a solid of the inhibitor of an elastase such as clavulanic acid, sulbactam, or tazobactam). In some embodiments, the particles are droplets of an inhibitor of an elastase such as clavulanic acid, sulbactam, or tazobactam dissolved in water or another liquid. In some embodiments, the inhibitor of an elastase such as clavulanic acid, sulbactam, or tazobactam is in a pharmaceutical composition comprising an antibiotic, for example, sosyn, augmentin, or Sultamicillin (Unasyn).

In some embodiments, the inhaled formulation includes an aerosol. In some embodiments, the inhaled formulation is delivered via a nebulizer. In some embodiments, the inhaled formulation is delivered to the lung of the respiratory system.

In some embodiments, the subject is not also being treated with amoxicillin or ticarcillin.

In some embodiments, the inhibitor of an elastase such as clavulanic acid, sulbactam, or tazobactam is delivered orally.

In one aspect, the invention features a pharmaceutical composition. The composition includes inhibitor of an elastase such as clavulanic acid, sulbactam, or tazobactam, and a pharmaceutically acceptable carrier, wherein the composition is substantially free of amoxicillin and ticarcillin.

In some embodiments, the method also includes administering an additional therapeutic agent, for example, an anti-infective or an anti-inflammatory agent. Exemplary antibiotics include gram negative, gram positive, and broad spectrum antibiotics. Antibiotics can be characterized into the following classifications. Aminoglycosides; beta-lactam antibiotics such as Carbacephems, Carbapenems, Cephalosporins, and Penicillins; Macrolides; antibiotic polypeptides; antibiotic lipopeptides; antibiotic glycopeptides; Monobactams; Quinolones; Sulfonamides; Tetracyclines; and others. Preferred antibiotics are those effective in treating gram negative infections. Exemplary preferred gram negative antibiotics include tobramycin and aztreonam. In some preferred embodiments, the antibiotic is formulated to provide desirable lung retention or residence time.

Exemplary antibiotics within the classes recited above are provided as follows. Exemplary Aminoglycosides include Streptomycin, Neomycin, Framycetin, Parpmycin, Ribostamycin, Kanamycin, Amikacin, Dibekacin, Tobramycin, Hygromycin B, Spectinomycin, Gentamicin, Netilmicin, Sisomicin, Isepamicin, Verdamicin, Amikin, Garamycin, Kantrex, Netromycin, Nebcin, and Humatin. Exemplary carbacephems include Loracarbef (Lorabid). Exemplary carbapenems include ertapenem, invanz, doripenem, finibax, imipenem/cilastatin, primaxin, meropenem, and merrem. Exemplary cephalosporins include cefadroxil, Duricef, cefazolin, ancef, cefalotin, cefalothin, keflin, cefalexin, Keflex, Cefaclor, Ceclor, Cefamandole, Mandole, Cefoxitin, Mefoxin, Cefprozill, Cefzil, Cefuroxime, Ceftin, Zinnat, Cefixime, Suprax, Cefdinir, Omnicef, Cefditoren, Spectracef, Cefoperazone, Cefobid, Cefotaxime, Claforan, Cefpodoxime, Fortaz, Ceftibuten, Cedax, Ceftizoxime, Ceftriaxone, Rocephin, Cefepime, Maxipime, and Ceftrobriprole. Exemplary glycopeptides include dalbavancin, oritavancin, Teicoplanin, Vancomycin, and Vancocin. Exemplary macrolides include Azithromycin, Sithromax, Smamed, Zitrocin, Clarithromycin, Biaxin, Dirithromycin, Erythromycin, Erythocin, Erythroped, Roxithromycin, Troleandomycin, Telithromycin, Ketek, and Spectinomycin. Exemplary monobactams include Aztreonam. Exemplary penicillins include Amoxicillin, Nvamox, Aoxil, Apicillin, Alocillin, Crbenicillin, Coxacillin, Diloxacillin, Flucloxacillin Floxapen, Mezlocillin, Meticillin, Nafcillin, Oacillin, Pnicillin, and Ticarcillin Exemplary polypeptides include Bacitracin, Colistin, and Polymyxin B. Exemplary quiniolones include Ciproflaxin, Cipro, Cproxin, Ciprobay, Enoxacin, Gatifloxacin, Tequin, Levofloxacin, Levaquin, Lomefloxacin, Moxifloxacin, Avelox, Norfloxacin, Noroxin, Ofloxacin, Ocuflox, Trovafloxacin, and Trovan. Exemplary sulfonamides include Mefenide, Prontosil (archaic), Sulfacetamide, Sulfamethizole, Sulfanilamide (archaic), Sulfasalazine, Sulfisoxazole, Trimethoprim, Trimethoprim-Sulfamethoxazole (co-trmoxazole), and Bactrim. Exemplary tetracyclines include Demeclocyline, Doxycycline, Vibramycin, Minocycline, Minocin, Oxytetracycline, Terracin, Tetracycline, and Sumycin. Other exemplary antibiotics include rshenamine, Salvarsan, Chloamphenicol, Chloromycetin, Clindamycin, Cleocin, Linomycin, Ethambutol, Fosfomycin, Fusidic Acid, Fucidin, Furazolidone, Isoniazid, Linezolid, Zyvox, Metronidazole, Flagyl, Mupirocin, Bactroban, Nitrofurantion, Macrodantin, Macrobid, Platensimycin, Pyrazinamide, Quinupristin/Dalfopristin, Syncerid, Rifampin (rifampicin), and Timidazole.

In one aspect, the invention features a composition for inhaled delivery to a subject, the composition including an inhibitor of elastase such as clavulanic acid, sulbactam, or tazobactam and a pharmaceutically acceptable carrier. In some embodiments, the composition is substantially free of amoxicillin and ticarcillin. In some embodiments, the inhibitor of an elastase such as clavulanic acid, sulbactam, or tazobactam is in a pharmaceutical composition comprising an antibiotic, for example, Zosyn, Augmentin, or Sultamicillin (Unasyn).

In some embodiments, the method also includes administering an additional therapeutic agent, for example, an anti-infective or an anti-inflammatory agent. Exemplary antibiotics include gram negative, gram positive, and broad spectrum antibiotics. Antibiotics can be characterized into the following classifications. Aminoglycosides; beta-lactam antibiotics such as Carbacephems, Carbapenems, Cephalosporins, and Penicillins; Macrolides; antibiotic polypeptides; antibiotic lipopeptides; antibiotic glycopeptides; Monobactams; Quinolones; Sulfonamides; Tetracyclines; and others. Preferred antibiotics are those effective in treating gram negative infections. Exemplary preferred gram negative antibiotics include tobramycin and aztreonam. In some preferred embodiments, the antibiotic is formulated to provide desirable lung retention or residence time.

Exemplary antibiotics within the classes recited above are provided as follows. Exemplary Aminoglycosides include Streptomycin, Neomycin, Framycetin, Parpmycin, Ribostamycin, Kanamycin, Amikacin, Dibekacin, Tobramycin, Hygromycin B, Spectinomycin, Gentamicin, Netilmicin, Sisomicin, Isepamicin, Verdamicin, Amikin, Garamycin, Kantrex, Netromycin, Nebcin, and Humatin. Exemplary carbacephems include Loracarbef (Lorabid). Exemplary carbapenems include ertapenem, invanz, doripenem, finibax, imipenem/cilastatin, primaxin, meropenem, and merrem. Exemplary cephalosporins include cefadroxil, Duricef, cefazolin, ancef, cefalotin, cefalothin, keflin, cefalexin, Keflex, Cefaclor, Ceclor, Cefamandole, Mandole, Cefoxitin, Mefoxin, Cefprozill, Cefzil, Cefuroxime, Ceftin, Zinnat, Cefixime, Suprax, Cefdinir, Omnicef, Cefditoren, Spectracef, Cefoperazone, Cefobid, Cefotaxime, Claforan, Cefpodoxime, Fortaz, Ceftibuten, Cedax, Ceftizoxime, Ceftriaxone, Rocephin, Cefepime, Maxipime, and Ceftrobriprole. Exemplary glycopeptides include dalbavancin, oritavancin, Teicoplanin, Vancomycin, and Vancocin. Exemplary macrolides include Azithromycin, Sithromax, Smamed, Zitrocin, Clarithromycin, Biaxin, Dirithromycin, Erythromycin, Erythocin, Erythroped, Roxithromycin, Troleandomycin, Telithromycin, Ketek, and Spectinomycin. Exemplary monobactams include Aztreonam. Exemplary penicillins include Amoxicillin, Nvamox, Aoxil, Apicillin, Alocillin, Crbenicillin, Coxacillin, Diloxacillin, Flucloxacillin Floxapen, Mezlocillin, Meticillin, Nafcillin, Oacillin, Pnicillin, and Ticarcillin Exemplary polypeptides include Bacitracin, Colistin, and Polymyxin B. Exemplary quiniolones include Ciproflaxin, Cipro, Cproxin, Ciprobay, Enoxacin, Gatifloxacin, Tequin, Levofloxacin, Levaquin, Lomefloxacin, Moxifloxacin, Avelox, Norfloxacin, Noroxin, Ofloxacin, Ocuflox, Trovafloxacin, and Trovan. Exemplary sulfonamides include Mefenide, Prontosil (archaic), Sulfacetamide, Sulfamethizole, Sulfanilamide (archaic), Sulfasalazine, Sulfisoxazole, Trimethoprim, Trimethoprim-Sulfamethoxazole (co-trmoxazole), and Bactrim. Exemplary tetracyclines include Demeclocyline, Doxycycline, Vibramycin, Minocycline, Minocin, Oxytetracycline, Terracin, Tetracycline, and Sumycin. Other exemplary antibiotics include rshenamine, Salvarsan, Chloamphenicol, Chloromycetin, Clindamycin, Cleocin, Linomycin, Ethambutol, Fosfomycin, Fusidic Acid, Fucidin, Furazolidone, Isoniazid, Linezolid, Zyvox, Metronidazole, Flagyl, Mupirocin, Bactroban, Nitrofurantion, Macrodantin, Macrobid, Platensimycin, Pyrazinamide, Quinupristin/Dalfopristin, Syncerid, Rifampin (rifampicin), and Timidazole.

In one aspect, the invention features a kit comprising a container and a composition comprising an inhibitor of elastase such as clavulanic acid, sulbactam, or tazobactam. In some embodiments, the composition is substantially free of amoxicillin and ticarcillin. The container provides for storing the composition and administering to a subject via inhalation.

In one aspect, the invention features a method of monitoring a subject being treated for a disorder characterized by unwanted serine protease activity, for example a disorder described herein. The method includes treating a subject with a composition comprising an inhibitor of elastase such as clavulanic acid, sulbactam, or tazobactam, and evaluating the subject for improvement in one or more parameters that can be indicative of improvement in the subjects condition. In some embodiments, the improvement is measured by evaluating the lung function of the subject (e.g., evaluating forced expiratory volume), evaluating an image of the lung (e.g., using X-ray, CT scan, or a magnetic resonance imaging agent such as He-3 or Xe-129), or evaluation of a surrogate marker such as a bacterial or other pathogenic marker, cell counts or measures of a viral titer or fluid (e.g., in the lung)). In some embodiments, the subject is evaluated using a CT scan.

### DETAILED DESCRIPTION

Methods of using an inhibitor of elastase such as clavulanic acid, sulbactam, or tazobactam are described herein. For example, the inventors have discovered that an inhibitor of elastase such as clavulanic acid, sulbactam, or tazobactam can be used to treat a disorder relating to a serine protease (e.g., Cathepsin G, elastase, and protease 3). In some embodiments, the disorder is characterized by unwanted serine protease activity. Exemplary methods of treatment include the treatment of conditions and disorders relating to excess elastase activity triggered by the recruitment of neutrophils to an inflammatory site and the release or leakage of excess elastase. In some preferred embodiments, the methods described herein include the treatment of cystic fibrosis or a symptom or complication thereof.

Elastase Inhibitors:

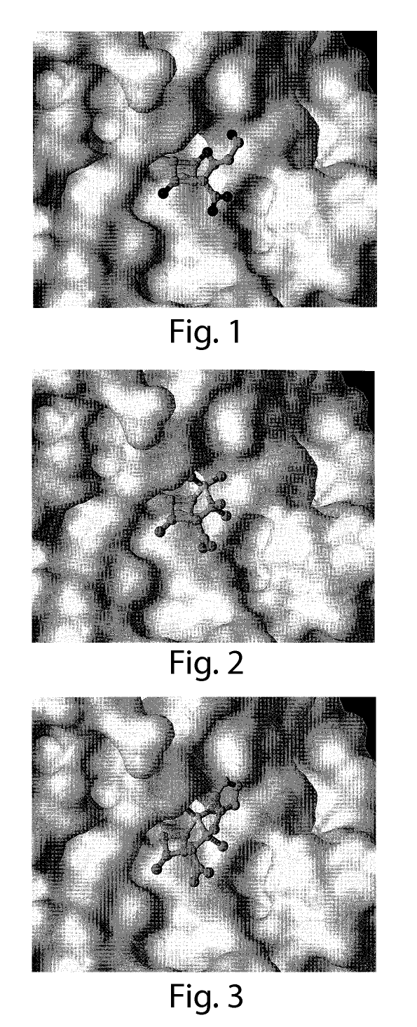

The inventors have discovered that clavulanic acid, sulbactam, and tazobactam bind to elastase. Models of clavulanic acid bound to elastase, sulbactam bound to elastase, and tazobactam bound to elastase are provide in FIGS. 1, 2, and 3 respectively. The binding of clavulanic acid is indirectly supported by the coordinates of the X-ray crystallographic structure of the reaction product of clavulanic acid bound to porcine pancreatic elastase. These coordinates are deposited in the Protein Data Bank (pdb.org), file designation 2IOT.

Clavulanic acid, also known as (2R,5R,Z)-3-(2-hydroxyethylidene)-7-oxo-4-oxa-1-aza-bicyclo[3.2.0]heptane-2-carboxylic acid, is a natural product isolated from Streptomyces clavuligerus. The structure of clavulanic acid is provided below:

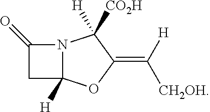

Sulbactam is also known as (2R,5R)-3,3-dimethyl-4,4,7-trioxo-4-thia-1-azabicyclo[3.2.0]heptane-2-carboxylic acid. The structure of sulbactam is provided below:

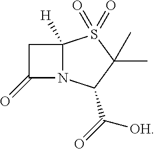

Tazobactam is also known as (2S,3S,5R)-3-methyl-4,4,7-trioxo-3-(triazol-1-ylmethyl)-4λ⁶-thia-1-azabicyclo[3.2.0]heptane-2-carboxylic acid. The structure of tazobactam is provided below:

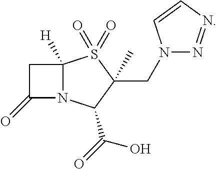

In some embodiments, an inhibitor of elastase such as clavulanic acid, sulbactam, or tazobactam is formed into a salt. An elastase inhibitor such as clavulanic acid, sulbactam, or tazobactam can be administered as a free acid, a zwitterion or as a salt. A salt can also be formed between a cation and a negatively charged substituent on a compound described herein. Suitable counterions include sodium ion, potassium ion, magnesium ion, calcium ion, and ammonium cations (e.g., a tetraalkyl ammonium cation such as tetramethylammonium ion). In compounds including a positively charged substituent, a salt can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate.

Salt forms of the compounds of any of the compounds described herein can be amino acid salts of carboxy groups (e.g. L-arginine, -lysine, -histidine salts). In some preferred embodiments, the inhibitor of elastase such as clavulanic acid, sulbactam, or tazobactam is formed as a lysine or arginine salt.

Pharmaceutically acceptable salts of the compounds of this invention also include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

Methods of Treatment:

An inhibitor of elastase such as clavulanic acid, sulbactam, or tazobactam can be administered to a subject, for example, a subject in need thereof, to treat a disorder characterized by unwanted serine protease activity, for example, unwanted activity of Cathepsin G, elastase, or Protease 3, e.g., unwanted elastase. Turino in “Chest 2002; 122; 1058-1060, reviews the history of the protease-antiprotease imbalance hypothesis, which is incorporated herein by reference. In some embodiments, the disorder treated with an elastase inhibitor is an inflammatory disorder or a disorder associated with the lung or respiratory system. Exemplary subjects include humans and other animals, e.g., livestock, pets, or animals bred for competition (e.g., racing).

An inhibitor of elastase such as clavulanic acid, sulbactam, or tazobactam can be administered prior to the onset of, at, or after the initiation of inflammation. When used prophylactically, an elastase inhibitor such as clavulanic acid, sulbactam, or tazobactam is preferably provided in advance of any inflammatory response or symptom. Administration of an inhibitor of elastase such as clavulanic acid, sulbactam, or tazobactam may attenuate inflammatory responses or symptoms. For example, when used prophylactically for a chronic condition, an inhibitor of elastase such as clavulanic acid, sulbactam, or tazobactam can be administered chronically to halt or control the damage resulting from the chronic condition. When used to control acute inflammatory exacerbations, for example, due to an infection, pollutants, allergies, etc, an inhibitor of elastase such as clavulanic acid, sulbactam, or tazobactam may be administered in conjunction with the condition, in response to the condition, or prophylactically.

Inhibitors of elastase can prevent the destruction of naturally occurring defense mechanisms. For example, an inhibitor of elastase can reduce the severity of an infection (e.g., a gram negative infection) and/or prevent the establishment of a chronic infection in, for example, a patient suffering from cystic fibrosis, COPD, emphysema or asthma. An inhibitor of elastase such as clavulanic acid, sulbactam, or tazobactam, and compositions described herein that include an inhibitor of elastase such as clavulanic acid, sulbactam, or tazobactam, can be used to treat an inflammatory disorder, for example, an inflammatory condition of the lung such as cystic fibrosis (for example, persistence of infection in a cystic fibrosis patient, for example pseudomonas or other gram negative infection), emphysema (for example, genetic AAT deficiency, COPD (e.g., lung degeneration in chronic bronchitis, lung degeneration in asthma, and other COPD), acute lung injury (ALI), acute respiratory distress syndrome (ARDS), or systemic inflammatory response syndrome (SIRS). In some embodiments, the condition is ALI and the ALI is caused by systemic inflammatory response syndrome (SIRS). In some embodiments, the disorder is a bronchial disorder such as bronchiectasis. In some embodiments, the disorder is a pulmonary disorder, for example, a chronic pulmonary disorder such as inflammation or cystic fibrosis. In some embodiments, the disorder is an acute pulmonary disorder, for example, including inflammation. In some embodiments, the acute pulmonary disorder is acute respiratory distress syndrome.

Cystic fibrosis is the most common inherited disease in white populations, having an incidence of about 1 in about 2500 newborns. Cystic fibrosis is generally managed with proactive treatment of airway infections as well as management of nutrition and lifestyle (e.g., maintaining an active lifestyle). Cystic fibrosis manifests in a plurality of organs, including, most notably, the upper and lower airways. Cystic fibrosis also manifests in the pancreas, bowel, and reproductive tracts. In general, the most likely cause of death in a subject suffering from cystic fibrosis is lung disease. Exemplary strategies for the management of cystic fibrosis, including the lung disease associated with cystic fibrosis, is described by Davies et al. in BMJ 2007; 335; 1255-1259, which is incorporated by reference herein. The nature and treatment of cystic fibrosis is also described by Cooley et al, in Journal of Leukocyte Biology, Vol. 83, April 2008, pp. 946-955, which is incorporated by reference herein.

Exemplary inflammatory conditions or conditions that may induce an inflammatory response include those that can be treated with the compounds and compositions described herein including, for example, asthma, multiple sclerosis, arthritis (e.g., rheumatoid arthritis, psoriatic arthritis, gouty arthritis, juvenile arthritis, or osteoarthritis), degenerative joint disease, spondouloarthropathies, systemic lupus erythematosus, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, an elastase inhibitor such as clavulanic acid, sulbactam, or tazobactam may be used to treat or prevent inflammatory exacerbations due to allergies or respiratory conditions, including asthma, bronchitis, bronchiectasis, pulmonary fibrosis, allergic rhinitis, oxygen toxicity, emphysema, chronic bronchitis, acute respiratory distress syndrome, and any chronic obstructive pulmonary disease (COPD). The compounds may be used to treat inflammatory exacerbations due to chronic hepatitis or HIV infection, including hepatitis B and hepatitis C.

In another embodiment, an elastase inhibitor such as clavulanic acid, sulbactam, or tazobactam can be used to treat a pulmonary disorder (e.g., a chronic or acute pulmonary disorder). In some embodiments, the pulmonary disorder includes an inflammatory component. Exemplary pulmonary disorders include acute lung injury (i.e., ALI), which can be caused, in some instances by systemic inflammatory response syndrome (i.e., SIRS), and acute respiratory distress syndrome (i.e., ARDS).

In some embodiments, an elastase inhibitor such as clavulanic acid, sulbactam, or tazobactam may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In some embodiments, an elastase inhibitor such as clavulanic acid, sulbactam, or tazobactam can be used to treat a disorder triggered by release of neutrophil elastase, for example, the release of neutrophil elastase in the pancreas. Exemplary disorders include pancreatitis.

In certain embodiments, an elastase inhibitor such as clavulanic acid, sulbactam, or tazobactam may be taken alone or in combination with other compounds useful for treating or preventing inflammation. Exemplary anti-inflammatory agents include, for example, steroids (e.g., Cortisol, cortisone, fludrocortisone, prednisolone (Medrol), 6[alpha]-methylprednisone, triamcinolone, betamethasone or dexamethasone), nonsteroidal antiinflammatory drugs (NSAIDS (e.g., aspirin, acetaminophen, tolmetin, ibuprofen, mefenamic acid, piroxicam, nabumetone, rofecoxib, celecoxib, etodolac or nimesulide).

In some embodiments, the method also includes administering an additional therapeutic agent, for example, an anti-infective or an anti-inflammatory agent. Exemplary antibiotics include gram negative, gram positive, and broad spectrum antibiotics. Antibiotics can be characterized into the following classifications. Aminoglycosides; beta-lactam antibiotics such as Carbacephems, Carbapenems, Cephalosporins, and Penicillins; Macrolides; antibiotic polypeptides; antibiotic lipopeptides; antibiotic glycopeptides; Monobactams; Quinolones; Sulfonamides; Tetracyclines; and others. Preferred antibiotics are those effective in treating gram negative infections. Exemplary preferred gram negative antibiotics include tobramycin and aztreonam. In some preferred embodiments, the antibiotic is formulated to provide desirable lung retention or residence time.

Exemplary antibiotics within the classes recited above are provided as follows. Exemplary Aminoglycosides include Streptomycin, Neomycin, Framycetin, Parpmycin, Ribostamycin, Kanamycin, Amikacin, Dibekacin, Tobramycin, Hygromycin B, Spectinomycin, Gentamicin, Netilmicin, Sisomicin, Isepamicin, Verdamicin, Amikin, Garamycin, Kantrex, Netromycin, Nebcin, and Humatin. Exemplary carbacephems include Loracarbef (Lorabid). Exemplary carbapenems include ertapenem, invanz, doripenem, finibax, imipenem/cilastatin, primaxin, meropenem, and merrem. Exemplary cephalosporins include cefadroxil, Duricef, cefazolin, ancef, cefalotin, cefalothin, keflin, cefalexin, Keflex, Cefaclor, Ceclor, Cefamandole, Mandole, Cefoxitin, Mefoxin, Cefprozill, Cefzil, Cefuroxime, Ceftin, Zinnat, Cefixime, Suprax, Cefdinir, Omnicef, Cefditoren, Spectracef, Cefoperazone, Cefobid, Cefotaxime, Claforan, Cefpodoxime, Fortaz, Ceftibuten, Cedax, Ceftizoxime, Ceftriaxone, Rocephin, Cefepime, Maxipime, and Ceftrobriprole. Exemplary glycopeptides include dalbavancin, oritavancin, Teicoplanin, Vancomycin, and Vancocin. Exemplary macrolides include Azithromycin, Sithromax, Smamed, Zitrocin, Clarithromycin, Biaxin, Dirithromycin, Erythromycin, Erythocin, Erythroped, Roxithromycin, Troleandomycin, Telithromycin, Ketek, and Spectinomycin. Exemplary monobactams include Aztreonam. Exemplary penicillins include Amoxicillin, Nvamox, Aoxil, Apicillin, Alocillin, Crbenicillin, Coxacillin, Diloxacillin, Flucloxacillin Floxapen, Mezlocillin, Meticillin, Nafcillin, Oacillin, Pnicillin, and Ticarcillin Exemplary polypeptides include Bacitracin, Colistin, and Polymyxin B. Exemplary quiniolones include Ciproflaxin, Cipro, Cproxin, Ciprobay, Enoxacin, Gatifloxacin, Tequin, Levofloxacin, Levaquin, Lomefloxacin, Moxifloxacin, Avelox, Norfloxacin, Noroxin, Ofloxacin, Ocuflox, Trovafloxacin, and Trovan. Exemplary sulfonamides include Mefenide, Prontosil (archaic), Sulfacetamide, Sulfamethizole, Sulfanilamide (archaic), Sulfasalazine, Sulfisoxazole, Trimethoprim, Trimethoprim-Sulfamethoxazole (co-trmoxazole), and Bactrim. Exemplary tetracyclines include Demeclocyline, Doxycycline, Vibramycin, Minocycline, Minocin, Oxytetracycline, Terracin, Tetracycline, and Sumycin. Other exemplary antibiotics include rshenamine, Salvarsan, Chloamphenicol, Chloromycetin, Clindamycin, Cleocin, Linomycin, Ethambutol, Fosfomycin, Fusidic Acid, Fucidin, Furazolidone, Isoniazid, Linezolid, Zyvox, Metronidazole, Flagyl, Mupirocin, Bactroban, Nitrofurantion, Macrodantin, Macrobid, Platensimycin, Pyrazinamide, Quinupristin/Dalfopristin, Syncerid, Rifampin (rifampicin), and Timidazole.

In another embodiment, the other therapeutic agent is a PDE4 inhibitor (e.g., roflumilast or rolipram). In another embodiment, the other therapeutic agent is an antihistamine (e.g., cyclizine, hydroxyzine, promethazine or diphenhydramine). In another embodiment, the other therapeutic agent is an anti-malarial (e.g., artemisinin, artemether, artsunate, chloroquine phosphate, mefloquine hydrochloride, doxycycline hyclate, proguanil hydrochloride, atovaquone or halofantrine). In one embodiment, the other therapeutic agent is drotrecogin alfa. In some embodiments, an elastase inhibitor such as clavulanic acid, sulbactam, or tazobactam is administered with an antibiotic for treating a gram negative infection, for example, an infection associated with cystic fibrosis. Exemplary antibiotics include azithromycin, cefaclor, ceftazidime, ciprofloxacin, gentamicin, imipenem, ofloxacin, piperacillin, and tobramycin.

Further examples of anti-inflammatory agents include, for example, aceclofenac, acemetacin, e-acetamidocaproic acid, acetaminophen, acetaminosalol, acetanilide, acetylsalicylic acid, S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzpiperylon, benzydamine, benzylmorphine, bermoprofen, betamethasone, betamethasone-17-valerate, bezitramide, [alpha]-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butorphanol, carbamazepine, carbiphene, caiprofen, carsalam, chlorobutanol, chloroprednisone, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide, cyclazocine, deflazacort, dehydrotestosterone, desomorphine, desonide, desoximetasone, dexamethasone, dexamethasone-21-isonicotinate, dexoxadrol, dextromoramide, dextropropoxyphene, deoxycorticosterone, dezocine, diampromide, diamorphone, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocoitolone, fluoresone, fluorometholone, fluperolone, flupirtine, fluprednidene, fluprednisolone, fluproquazone, flurandrenolide, flurbiprofen, fluticasone, formocortal, fosfosal, gentisic acid, glafenine, glucametacin, glycol salicylate, guaiazulene, halcinonide, halobetasol, halometasone, haloprednone, heroin, hydrocodone, hydro cortamate, hydrocortisone, hydrocortisone acetate, hydrocortisone succinate, hydrocortisone hemisuccinate, hydrocortisone 21-lysinate, hydrocortisone cypionate, hydromorphone, hydroxypethidine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, mazipredone, meclofenamic acid, medrysone, mefenamic acid, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate, myrophine, nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papavereturn, paramethasone, paranyline, parsalmide, pentazocine, perisoxal, phenacetin, phenadoxone, phenazocine, phenazopyridine hydrochloride, phenocoll, phenoperidine, phenopyrazone, phenomorphan, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, viminol, xenbucin, ximoprofen, zaltoprofen and zomepirac.

In some embodiments, an elastase inhibitor such as clavulanic acid, sulbactam, or tazobactam can be administered with a bronchial dilator. Exemplary bronchial dilators include long acting and short acting agents, e.g., vephylline, ephedra,

In some embodiments, an elastase inhibitor such as clavulanic acid, sulbactam, or tazobactam can be administered to a subject who is also being administered oxygen therapy (e.g., chronic oxygen therapy).

Pharmaceutical Compositions/Routes of Administration:

An elastase inhibitor such as clavulanic acid, sulbactam, or tazobactam can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 3 to 170 hours, or according to the requirements of the particular drug. In some preferred embodiments, an elastase inhibitor such as clavulanic acid, sulbactam, or tazobactam is administered by inhalation. Typically, an elastase inhibitor such as clavulanic acid, sulbactam, or tazobactam will be administered from about 1 to about 8 times per day. Such administration can be used as a chronic or acute therapy. The amount of an elastase inhibitor such as clavulanic acid, sulbactam, or tazobactam that may be combined with the carrier materials to produce a single dosage form will vary depending upon the host treated and the particular mode of administration. A typical preparation will contain from about 5% to about 99.9% an elastase inhibitor such as clavulanic acid, sulbactam, or tazobactam (w/w). Alternatively, such preparations contain from about 20% to about 80% an elastase inhibitor such as clavulanic acid, sulbactam, or tazobactam.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of an elastase inhibitor such as clavulanic acid, sulbactam, or tazobactam may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent or chronic treatment on a long-term basis upon any recurrence of disease symptoms.

The compositions delineated herein include an elastase inhibitor such as clavulanic acid, sulbactam, or tazobactam, as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts (e.g., buffered saline) or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein. In some preferred embodiments, the carrier is water or buffered saline.

The pharmaceutical compositions of this invention may be administered orally, parenterally, by inhalation spray, nebulizer, topically, rectally, nasally, buccally, vaginally or via an implanted reservoir, preferably by inhalation administration or oral administration. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by aerosol, nebulizer, or inhalation. In some embodiments, the composition is in the form of a dry powder, a suspension, or a solution. In some preferred embodiments, the composition is formulated to provide deep lung penetration of the inhibitor of elastase. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Exemplary methods and devices for aerosol or inhalation include those described in U.S. Pat. No. 6,962,151, which is incorporated herein by reference in its entirety. In some preferred embodiments, the elastase inhibitor such as clavulanic acid, sulbactam, or tazobactam is administered by inhalation.

Compositions formulated for inhaled delivery generally include particles having a mean diameter of from about 0.1 μm to about 50 μm (e.g., from about 0.1 μm to about 10 μm, or from about 0.2 μm to about 5 μm. In some embodiments, the composition includes a dispersion of suitably-sized dry particles, for example, precipitants or crystals) or a dispersion of a solution (e.g., droplets) of a suitable size.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Kits:

An elastase inhibitor such as clavulanic acid, sulbactam, or tazobactam can be provided in a kit. The kit includes (a) an elastase inhibitor such as clavulanic acid, sulbactam, or tazobactam, and, optionally (b) informational material. In some embodiments, the kit includes a container suitable for dispersing the elsastase inhibitor. Exemplary containers include a nebulizer or an aerosol device for inhalation.

The informational material can be descriptive, instructional, marketing or other material that relates to the methods described herein and/or the use of an elastase inhibitor such as clavulanic acid, sulbactam, or tazobactam for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer an elastase inhibitor such as clavulanic acid, sulbactam, or tazobactam in a suitable manner to perform the methods described herein, e.g., in a suitable dose, dosage form, or mode of administration (e.g., a dose, dosage form, or mode of administration described herein). In another embodiment, the informational material can include instructions to administer an elastase inhibitor such as clavulanic acid, sulbactam, or tazobactam to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to an elastase inhibitor such as clavulanic acid, sulbactam, or tazobactam, the composition of the kit can include other ingredients, such as a solvent or buffer, a stabilizer, a preservative, a flavoring agent (e.g., a bitter antagonist or a sweetener), a fragrance or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

An elastase inhibitor such as clavulanic acid, sulbactam, or tazobactam can be provided in any form, e.g., liquid, dried, lyophilized, a dry powder, or crystalline form. In some embodiments, the elastase inhibitor is provided as a dispersed material. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit. In some embodiments, the elastase inhibitor such as clavulanic acid, sulbactam, or tazobactam can be delivered as a dry powder (e.g., wherein the powder includes particles of a suitable size).

The kit can include one or more containers for the composition containing an elastase inhibitor such as clavulanic acid, sulbactam, or tazobactam. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

In some preferred embodiments, the kit includes a nebulizer or an aerosol dispenser, for example, which provides an inhaled elastase inhibitor such as clavulanic acid, sulbactam, or tazobactam to the subject.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

1. A method of treating a disorder in a subject, the method comprising administering to a subject an elastase inhibitor such as clavulanic acid, sulbactam, or tazobactam.

2. The method of claim 1, wherein the subject is not being treated with amoxicillin or ticarcillin.

3. The method of claim 1, wherein the disorder is a disorder related to excess elastase activity.

4. The method of claim 1, wherein the disorder is an inflammatory disorder.

5. The method of claim 4, wherein the inflammatory disorder is an inflammatory condition of the lung.

6. The method of claim 5, wherein the inflammatory condition of the lung is selected from the group consisting of: COPD, emphysema, cystic fibrosis, acute lung injury (ALI), acute respiratory distress syndrome (ARDS), bronchitis, and asthma (e.g., chronic asthma).

7. The method of claim 5, wherein the condition is ALI and the ALI is caused by systemic inflammatory response syndrome (SIRS).

8. The method of claim 1, wherein the disorder is a bronchial disorder.

9. The method of claim 8, wherein the bronchial disorder is bronchiectasis.

10. The method of claim 1, wherein the disorder is a pulmonary disorder.

11. The method of claim 10, wherein the pulmonary disorder is a chronic pulmonary disorder.

12. The method of claim 11, wherein the chronic pulmonary disorder comprises inflammation.

13. The method of claim 11, wherein the chronic pulmonary disorder is cystic fibrosis.

14. The method of claim 11, wherein the pulmonary disorder is an acute pulmonary disorder.

15. The method of claim 14, wherein the acute pulmonary disorder comprises inflammation.

16. The method of claim 14, wherein the acute pulmonary disorder is acute respiratory distress syndrome.

17-20. (canceled)

21. A method of treating a condition or disorder triggered by a neutrophil response, the method comprising administering to a subject an elastase inhibitor such as clavulanic acid, sulbactam, or tazobactam.

22-32. (canceled)

33. A pharmaceutical composition, the composition comprising an elastase inhibitor such as clavulanic acid, sulbactam, or tazobactam, and a pharmaceutically acceptable carrier, wherein the composition is substantially free of amoxicillin and ticarcillin.

34-39. (canceled)

40. A composition for inhaled delivery to a subject, the composition comprising an elastase inhibitor such as clavulanic acid, sulbactam, or tazobactam and a pharmaceutically acceptable carrier.

41-46. (canceled)
